(19) United States
(12) Patent Application Publication
(10) Pub. No.: US 2023/0053880 A1
(43) Pub. Date: Feb. 23, 2023

(54) OPTIMIZATION APPARATUS, OPTIMIZATION METHOD, AND PROGRAM

(71) Applicant: NIPPON TELEGRAPH AND TELEPHONE CORPORATION, Tokyo (JP)

(72) Inventors: Takashi ISEZAKI, Musashino-shi, Tokyo (JP); Mana SASAGAWA, Musashino-shi, Tokyo (JP); Yoshiki NISHIKAWA, Musashino-shi, Tokyo (JP)

(73) Assignee: NIPPON TELEGRAPH AND TELEPHONE CORPORATION, Tokyo (JP)

(21) Appl. No.: 17/790,725

(22) PCT Filed: Jan. 10, 2020
(86) PCT No.: PCT/JP2020/000678

§ 371 Date: Jul. 2, 2022

(51) Int. Cl. G16H 50/50

(52) U.S. Cl. CPC G16H 50/50

(57) ABSTRACT

A technology for simulating physiological activity more appropriately is provided. An optimization apparatus according to an embodiment includes an estimation unit, a first calculation unit, a second calculation unit, and an updating unit. The estimation unit estimates a physiological parameter indicating a physiological state using an estimation model. The first calculation unit calculates a first difference value based on a first estimated value of the physiological parameter in a first time interval and an actually measured value of the physiological parameter in the first time interval. The second calculation unit calculates a second difference value based on the first estimated value and a second estimated value of the physiological parameter in a second time interval previous to the first time interval. The updating unit adjusts the estimation model based on the first difference value and the second difference value.

### TECHNICAL FIELD

Embodiments of the present disclosure relate to an optimization apparatus, an optimization method, and a program.

### BACKGROUND ART

The bodies of animals, for example, human bodies, are configured through activities of various organs. When the activity of each organ can be ascertained in detail, portions in bad condition can be identified. Accordingly, technologies for accurately estimating the activity of each organ in detail are useful.

Construction of a simulator of the activity of a kidney has been attempted in order to understand the function of the kidney (for example, see NPL 1).

### CITATION LIST

### Non Patent Literature

[NPL 1] Jan Silar et al., “Model visualization for e-learning, Kidney simulator for medical students,” in Proceedings of the 13th International Modelica Conference, Regensburg, Germany, Mar. 4 to 6, 2019, 2019, vol. 157, pp. 393 to 402

### SUMMARY OF THE INVENTION

### Technical Problem

However, it is not easy to accurately simulate the physiological activity of an organ that occurs in vivo. Therefore, simulated activity of the related art deviates from actual physiological activity.

The present disclosure has been devised in view of the foregoing circumstances, and an object of the present disclosure is to provide a technology for simulating physiological activity more appropriately.

### Means for Solving the Problem

To solve the foregoing problem, a first aspect of the present disclosure is an optimization apparatus including an estimation unit configured to estimate a physiological parameter indicating a physiological state using an estimation model; a first calculation unit configured to calculate a first difference value based on a first estimated value of the physiological parameter in a first time interval and an actually measured value of the physiological parameter in the first time interval; a second calculation unit configured to calculate a second difference value based on the first estimated value and a second estimated value of the physiological parameter in a second time interval previous to the first time interval; and an updating unit configured to adjust the estimation model based on the first difference value and the second difference value.

### Effects of the Invention

According to the first aspect of the present disclosure, the estimation model is adjusted in consideration of a change in the estimated value of the physiological parameter in a given time interval, in addition to a difference between the actually measured value and the estimated value of the physiological parameter using the estimation model. Use of the estimation model adjusted in this way makes it possible to perform simulation that better reflects physiological activity of an individual subject.

That is, according to the present disclosure, it is possible to provide a technology for simulating physiological activity more appropriately.

### BRIEF DESCRIPTION OF DRAWINGS

FIG. 1 is a diagram illustrating a functional configuration of an optimization apparatus according to an embodiment of the present disclosure.

FIG. 2 is a block diagram illustrating a hardware configuration of the optimization apparatus according to the embodiment of the present disclosure.

FIG. 3 is a flowchart illustrating an overview of entire processing in the optimization apparatus illustrated in FIG. 1.

FIG. 4 is a flowchart illustrating details of simulation processing in the optimization apparatus illustrated in FIG. 1.

FIG. 5 is a diagram illustrating an example of sample data.

FIG. 6 is a diagram illustrating examples of initial viscera parameters.

FIG. 7 is a diagram illustrating an example of an action list of a user.

FIG. 8 is a diagram illustrating an example of a simulation result of a blood sodium concentration in 0 to 24 hours.

FIG. 9 is a diagram illustrating an example of a simulation result of a blood sodium concentration in 24 to 48 hours.

FIG. 10 is a diagram illustrating an example of a simulation result for each target time.

FIG. 11 is a diagram illustrating an example of a search region used to optimize viscera parameters.

### DESCRIPTION OF EMBODIMENTS

Hereinafter, embodiments of the present disclosure will be described with reference to the drawings. The same or similar reference numerals will be given to elements which are the same as or similar to the elements that have already been described, and repeated description thereof will essentially be omitted. For example, when there are a plurality of the same or similar elements, and these elements are not distinguished, common reference numerals may be used to describe them. To distinguish the elements from each other in the description, branch numbers may be used in addition to the common reference numerals.

### Embodiment

### Configuration

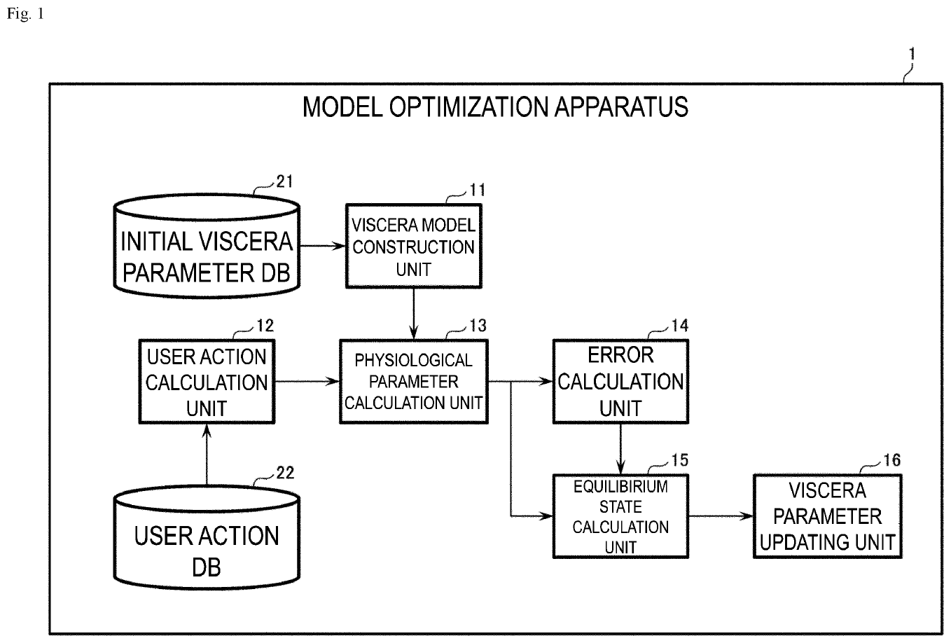

FIG. 1 illustrates an example of a functional configuration of a model optimization apparatus 1 serving as an optimization apparatus according to an embodiment of the present disclosure. The model optimization apparatus 1 optimizes a model for simulating activity of an organ, in particular, an internal organ, and is configured by, for example, a personal computer or a server computer.

The bodies of animals including humans perform intake and discharge of solids, liquids, and gases. Intake substances are absorbed in each organ in vivo and are discharged. To observe an operation of each organ in vivo, it is necessary to consider an input and an output in each organ and intake and discharge in the body.

Hereinafter, a quantitative value of a solid, a liquid, or a gas taken into and discharged by a human or animal body or each organ in vivo is referred to as a “physiological parameter.” For expiratory air, examples of the physiological parameter include an oxygen concentration and a carbon dioxide concentration in the expiratory air. For blood circulated in vivo, examples of the physiological parameter include a blood volume and a blood component (sodium concentration or the like).

In each organ in vivo, an output is changed in accordance with the volume and quality of an input substance and an activity characteristic of each organ. For example, for a kidney, a concentration of sodium to be discharged is adjusted in accordance with a blood sodium concentration. When a kidney has an activity characteristic in which an output is defined with a specific threshold, this threshold is a parameter indicating the activity characteristic of the kidney. Here, a parameter used to describe activity characteristics of each organ are referred to as a “viscera parameter.” The viscera parameter also serves as an adjustable parameter of a mathematical model of a viscera function and may be also referred to as a “model parameter.”

The model optimization apparatus 1 includes an initial viscera parameter database (DB) 21, a user action DB 22, a viscera model construction unit 11, a user action calculation unit 12, a physiological parameter calculation unit 13, an error calculation unit 14, an equilibrium state calculation unit 15, and a viscera parameter updating unit 16.

The initial viscera parameter DB 21 retains a preset initial viscera parameter used as an initial value of the foregoing viscera parameter.

The user action DB 22 stores a predefined user action list of actions of a user who is a subject.

The viscera model construction unit 11 constructs a mathematical model of viscera activity of a heart or the like based on the initial viscera parameter stored in the initial viscera parameter DB 21.

The user action calculation unit 12 calculates a user action based on the user action list stored in the user action DB 22 and calculates an intake substance volume and a discharge substance volume of the user.

The physiological parameter calculation unit 13 serving as an estimation unit receives the mathematical model constructed by the viscera model construction unit 11 and the calculation result calculated by the user action calculation unit 12, and calculates an estimated value of the physiological parameter.

The error calculation unit 14 serving as a first calculation unit receives the calculation result calculated by the physiological parameter calculation unit 13 and calculates an error between the simulation result and sample data.

The equilibrium state calculation unit 15 serving as a second calculation unit receives the calculation result calculated by the physiological parameter calculation unit 13 and the calculation result calculated by the error calculation unit 14, calculates a numerical value related to an equilibrium state (hereinafter referred to as an “equilibrium state”), adds the numerical value and the error, and outputs a value indicating the degree of inappropriateness of the simulation.

The viscera parameter updating unit 16 serving as an updating unit optimizes the viscera parameter based on the degree of inappropriateness output from the equilibrium state calculation unit 15.

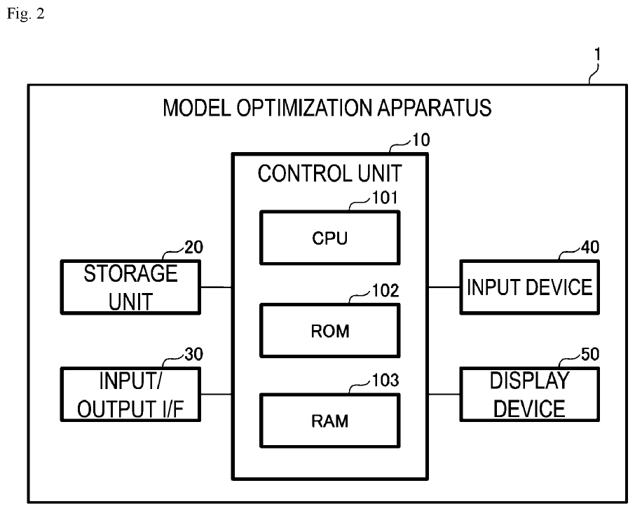

FIG. 2 illustrates an example of a hardware configuration of the foregoing model optimization apparatus 1.

In this example, the model optimization apparatus 1 includes a control unit 10, a storage unit 20, an input/output interface (I/F) 30, an input device 40, and a display device 50.

The control unit 10 includes a central processing unit (CPU) 101, a read-only memory (ROM) 102, and a random access memory (RAM) 103 and controls each constituent element. The CPU 101 is an example of a hardware processor. The CPU 101 loads a program stored in the storage unit 20 to the RAM 103. When the CPU 101 executes the program, the control unit 10 functions as the viscera model construction unit 11, the user action calculation unit 12, the physiological parameter calculation unit 13, the error calculation unit 14, the equilibrium state calculation unit 15, and the viscera parameter updating unit 16 described above. The CPU 101 may be substituted with an MPU, a GPU, an ASIC, an FPGA, or the like. The CPU 101 may be a single CPU or a plurality of CPUs.

The storage unit 20 is, for example, an auxiliary storage device such as a hard disk drive (HDD), a solid-state drive (SSD), or a semiconductor memory (for example, a flash memory). The storage unit 20 stores, in a non-transitory manner, a program to be executed by the control unit 10, setting data necessary to execute the program, and the like. The storage unit 20 includes the initial viscera parameter DB 21 and the user action DB 22 as storage areas necessary to achieve the embodiment. A storage medium included in the storage unit 20 is a medium that accumulates information of a stored program or the like electronically, magnetically, optically, mechanically, or chemically so that the information of the stored program can be read by a computer, another device, a machine, or the like. Several programs may be stored in the ROM 102.

The input/output interface (I/F) 30 includes, for example, one or more wired or wireless communication interface units and enables transmission and reception of information with an external device. For example, a wired LAN is used as the wired interface. For example, an interface in which a low-power wireless data communication standard such as a wireless LAN or Bluetooth (trade name) is adopted is used as the wireless interface. The input/output interface 30 may include a USB port.

The input device 40 is, for example, a keyboard, a mouse, a touch screen, a button, or a switch and receives an operation on the model optimization apparatus 1 from a user.

The display device 50 is, for example, a liquid crystal or organic electro-luminescence (EL) display or a speaker and displays display data generated by the control unit 10.

As the input device 40 and the display device 50, devices embedded in the model optimization apparatus 1 may be used or an input device and an output device of another information terminal which can communicate via a network may be used.

In the specific hardware configuration of the model optimization apparatus 1, constituent elements can be appropriately omitted, substituted, or added according to the embodiment. For example, the control unit 10 may include a plurality of processors.

In physiological activity of a human or animal body, each physiological parameter such as a nutrient or hormone is considered to be maintained in an equilibrium state, that is, in a state in which each physiological parameter converges within a given range despite a slight increase or decrease for a specific period of time. As described above, it is difficult to accurately simulate physiological activity of a viscera system that occurs in vivo. However, it is conceivable that physiological activity of an individual can be simulated more appropriately by incorporating the equilibrium state into a simulation. Considering this point, the model optimization apparatus 1 according to an embodiment optimizes a model in consideration of an equilibrium state of a physiological parameter due to an operation of viscera or an action of a subject.

In an example described below, a physiological parameter is a blood sodium concentration, and the model optimization apparatus 1 constructs an estimation model in which the equilibrium state of the blood sodium concentration is incorporated. Here, needless to say, the embodiments are not limited to the physiological parameter of the blood sodium concentration.

### Operation

Next, an operation of the model optimization apparatus 1 will be described.

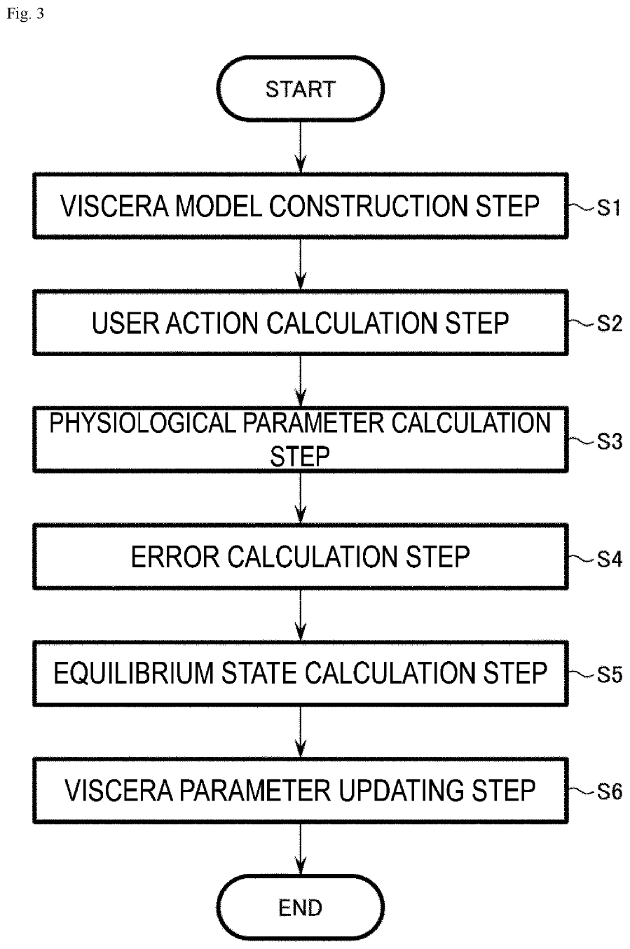

FIG. 3 illustrates an example of an overview of an entire operation of the model optimization apparatus 1. According to the embodiment, an operation of the model optimization apparatus 1 includes a viscera model construction step S1, a user action calculation step S2, a physiological parameter calculation step S3, an error calculation step S4, an equilibrium state calculation step S5, and a viscera parameter updating step S6.

In the viscera model construction step S1, the viscera model construction unit 11 performs mathematical modeling of a viscera function which is a simulation target.

In the user action calculation step S2, the user action calculation unit 12 performs calculation of a user action based on a pre-defined action list of the user at each time and calculates an intake or discharge substance volume of the user.

In the physiological parameter calculation step S3, the physiological parameter calculation unit 13 updates the physiological parameter based on the mathematical model constructed in the viscera model construction step S1, the intake or discharge substance volume calculated in the user action calculation step S2, and the current physiological parameter.

In the error calculation step S4, the error calculation unit 14 calculates, as an error, a difference between sample data which is an actually measured value measured from the user and an estimated value calculated by simulation.

In the equilibrium state calculation step S5, the equilibrium state calculation unit 15 calculates, as an equilibrium state, a difference in change characteristics of the physiological parameter between a target time interval and an immediately previous target time interval. Here, the terms “time interval,” “period,” and “period of time” are used interchangeably.

In the viscera parameter updating step S6, the viscera parameter updating unit 16 selects an optimum viscera parameter based on the calculated error and equilibrium state.

Next, the details of the operation of the model optimization apparatus 1 will be described giving a specific example.

The model optimization apparatus 1 optimizes the model based on the sample data measured from each user. In the optimization, the model optimization apparatus 1 acquires the viscera parameter based on viscera activity of each user by constructing the model into which the physiological equilibrium state of each user is incorporated.

First, it is assumed that sample data of daily intake calories [kcal/day] and blood sodium concentration [mEq/L] is acquired from two users (users A and B).

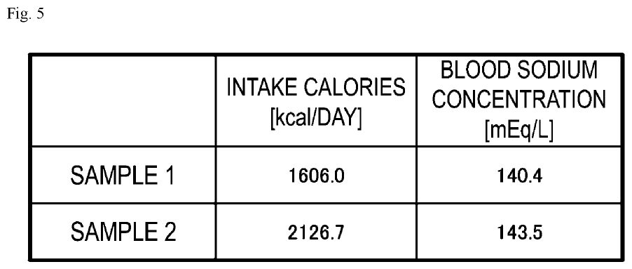

FIG. 5 illustrates examples of the sample data. Sample 1 indicates data measured from the user A and has values of intake calories of 1606.0 [kcal/day] and a blood sodium concentration of 140.4 [mEq/L]. Similarly, Sample 2 indicates data measured from the user B and has values of intake calories of 2126.7 [kcal/day] and a blood sodium concentration of 143.5 [mEq/L]. In the embodiment, the data is initial values of the simulation and serves as a reference of error calculation of the simulation.

The viscera model construction unit 11 reads the initial viscera parameter from the initial viscera parameter DB 21 and constructs the mathematical model of viscera activity in the foregoing viscera model construction step S1. The blood sodium concentration is simulated by modeling an increase in concentration through salt intake and a decrease in concentration through blood sodium removal in a kidney.

Here, a blood sodium volume removed in the kidney is determined based on a blood sodium concentration. The viscera model construction unit 11 according to the embodiment performs the modeling of discharged sodium volume DSV [mEq] as in the following equation.

$$D S V = \frac{D S {V}_{vol}}{1 + {e}^{- D S {V}_{vel} ( B S C - B B S C )}} \qquad [ Math . 1 ]$$

In the foregoing equation, BSC indicates a current blood sodium concentration and BBSC indicates a base blood sodium concentration. The base blood sodium concentration is, for example, a target blood sodium concentration of the kidney of each user. In the example, the base blood sodium concentration BBSC is assumed to be 140 [mEq/L].

In the foregoing equation, DSVᵥₒₗ and DSVᵥₑₗ are viscera parameters which are optimization targets and values maintained in the initial viscera parameter DB 21 are set first. DSVᵥₒₗ is a parameter related to a maximum amount (volume) in the foregoing equation and DSVᵥₑₗ is a parameter related to a change speed (velocity) of a change in the value of the blood sodium concentration BSC at each time.

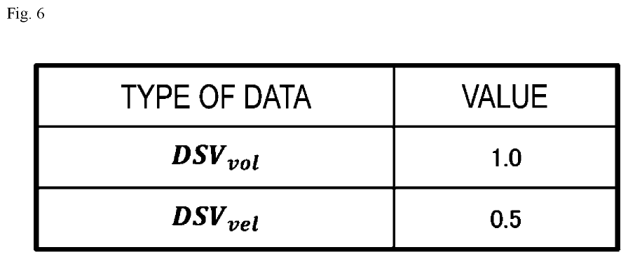

FIG. 6 illustrates examples of specific values of the initial viscera parameters DSVᵥₒₗ and DSVᵥₑₗ maintained in the initial viscera parameter DB 21. In this example, the initial parameters of DSVᵥₒₗ and DSVᵥₑₗ are set to 1.0 and 0.5, respectively.

The viscera model construction unit 11 outputs the foregoing calculation equation of the DSV as a viscera model to the physiological parameter calculation unit 13. The viscera model construction unit 11 outputs all calculation equations to the physiological parameter calculation unit 13 when a plurality of viscera functions are modeled.

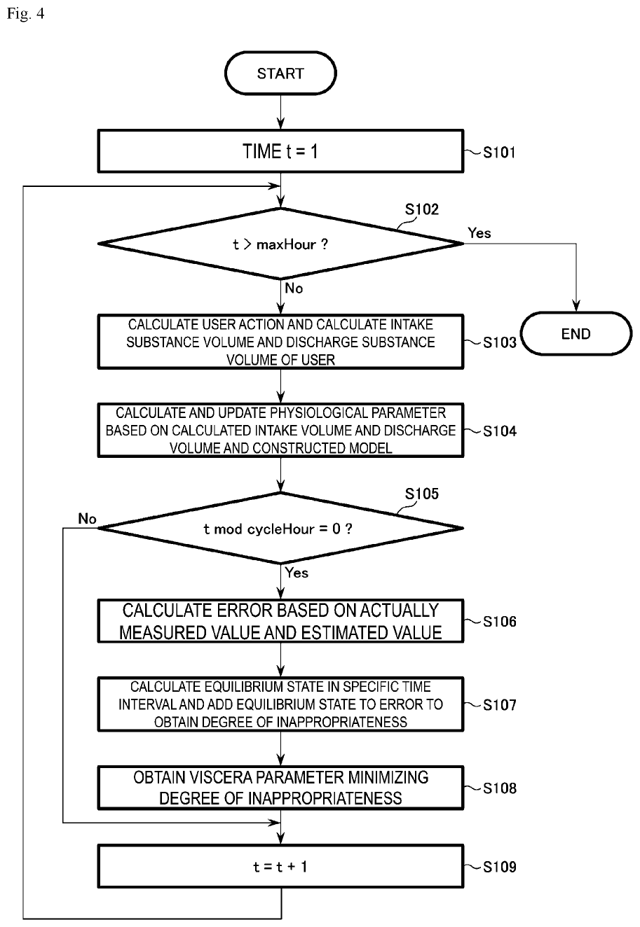

Of the processing performed by the model optimization apparatus 1, FIG. 4 illustrates an example of simulation processing performed by using the constructed viscera model.

In FIG. 4, maxHour and cycleHour are calculation time and time span at which an equilibrium state are calculated in the simulation, respectively and are assumed to be 72 hours and 24 hours in the example, respectively. Here, it is assumed that the calculation is performed when a time t is a cycle of cycleHour. In the example, since cycleHour is 24, the calculation is performed when tis 24, 48, or 72.

In step S101 of FIG. 4, the control unit 10 of the model optimization apparatus 1 sets the time t to an initial value in the simulation (t=1).

In step S102, the control unit 10 of the model optimization apparatus 1 determines whether the time t exceeds maxHour (in this example, 72 hours). When t>maxHour is satisfied (YES), the processing ends. When t>maxHour is not satisfied (NO), the subsequent processing continues.

In step S103, the user action calculation unit 12 calculates the user action based on the user action list read from the user action DB 22 and calculates an intake substance volume and a discharge substance volume of the user. Here, it is assumed that salt intake is performed by a dietary intake action and the dietary intake action is calculated based on the user action list.

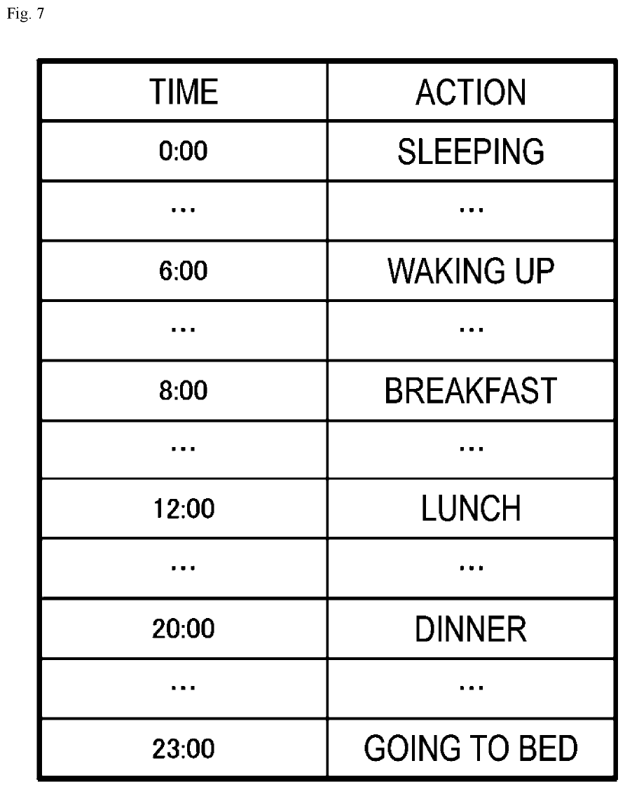

FIG. 7 illustrates an example of a chronological action list of the user stored in the user action DB 22.

In this example, the user action list includes actions such as sleeping at 0:00, waking up at 6:00, breakfast at 8:00, lunch at 12:00, dinner at 20:00, and going to bed at 23:00. Accordingly, the user action calculation unit 12 calculates sodium chloride volumes as intake substances in the actions of breakfast, lunch, and dinner in the user action list. In the flowchart of FIG. 4, the user action calculation unit 12 performs calculation of the meal intake actions when a remainder of time 24/t (hereinafter referred to as “t mod 24”) is 8, 12, or 20.

It can be estimated that an intake volume of salt, that is, sodium chloride, is proportional to an intake volume of food. In the embodiment, for example, a coefficient of proportionality is assumed to be 0.004 [g/kcal]. In this case, an intake sodium chloride volume [g/day]=intake calories [kcal/day]×0.004 [g/kcal] is obtained. An intake sodium chloride volume of the user A is 1606.0×0.004=6.424 [g/day] and an intake sodium chloride volume of the user B is 2126.7×0.004=8.5068 [g/day].

When the meal intake volumes in three meals of breakfast, lunch, and dinner in a day are assumed to be equal every time as in the action list illustrated in FIG. 7, an intake sodium chloride volume INCL of each meal is calculated as follows:

INCL_{userA}=6.424/3=2.14 [g] for the user A; and

INCL_{userB}=8.5068/3=2.8356 [g] for the user B.

The user action calculation unit 12 outputs the calculated intake sodium chloride volumes to the physiological parameter calculation unit 13. When the user action calculation unit 12 calculates other intake substance volumes or discharge substance volumes, all the calculation results are output to the physiological parameter calculation unit 13.

Information used for the user action calculation unit 12 to perform the calculation from the action list is not limited to the number of meals and the time. For example, the user action calculation unit 12 can further include a sleeping action in the calculation. When the action list further includes information regarding various exercises (for example, walking, running, and going up stairs), the user action calculation unit 12 may include the exercises in the calculation. More appropriate simulation can be performed by adding up discharged water in consideration of sweating in exercise or sleep.

In step S104, the physiological parameter calculation unit 13 calculates the physiological parameter indicating physiological activity by using, as inputs, the calculation equation of DSV received from the viscera model construction unit 11 and all the calculation results received from the user action calculation unit 12. More specifically, in the example, the physiological parameter calculation unit 13 performs calculation of the blood sodium concentration based on the intake sodium chloride volume calculated by the user action calculation unit 12 and updating of the blood sodium concentration based on the DSV calculation.

(S104-1) Calculation of Blood Sodium Concentration Based on Intake Sodium Chloride Volume

The physiological parameter calculation unit 13 first calculates the blood sodium concentration based on the intake sodium chloride volume. As described above, when the time (t mod 24) is 8, 12, or 20, the intake sodium chloride volume calculated by the user action calculation unit 12 is added.

When the sodium volume IN is calculated from the sodium chloride volume, an atomic weight 23 of Na and an atomic weight 35.5 of Cl can be used in the following equation.

IN=INCL×23/58.5

INCL is the intake sodium chloride volume obtained in step S103. INCL_{userA} is 2.14 [g] for the user A and INCL_{userB} is 2.8356 [g] for the user B.

Here, the intake sodium is assumed to be all dissolved in blood. A blood sodium concentration increase volume IBSC [mEq/L] due to the dissolution is obtained using the following equation.

IBSC=INCL×1000/10/2.3/BV

In the foregoing equation, BV is a blood volume and is assumed to be 5.0 [L] for both of the users A and B in the example. With the foregoing equation, the blood sodium concentration increase volumes IBSC [mEq/L] of the users A and B can be obtained as follows:

IBSC_{userA}=18.608 [mEq/L]

IBSC_{userB}=24.657 [mEq/L]

Accordingly, blood sodium concentrations I′BSC [mEq/L] to which the blood sodium concentration increase volumes are added are as follows:

I′BSC_{userA}=140.4+18.608=159.008 [mEq/L]; and

I′BSC_{userA}=143.5+24.657=168.157 [mEq/L].

(S104-2) Updating of Blood Sodium Concentration Based on Calculation of Discharge Sodium DSV

Subsequently, the physiological parameter calculation unit 13 updates the blood sodium concentration based on the discharge sodium DSV calculation, irrespective of the presence or absence of an action of the user action list. When other viscera functions are modeled, all the viscera functions are calculated every time in the simulation.

When the above-described BBSC=140, DSVᵥₒₗ=1, and DSVᵥₑₗ=0.5 are used in the foregoing DSV calculation equation, the following equation can be obtained. In the example, the physiological parameter calculation unit 13 performs the DSV calculation for each user using the following equation.

$$D S V = \frac{1}{1 + {e}^{- 0.5 ( B S C - 140 )}} \qquad [ Math . 2 ]$$

For example, for the user A, calculation is performed as in the following equation and DSV_{userA}=0.999925453 [mEq/L] is obtained.

$$D S {V}_{userA} = \frac{1}{1 + {e}^{- 0.5 ( 159.008 - 140 )}} \qquad [ Math . 3 ]$$

For the user B, similar calculation is performed and a discharge sodium volume DSV_{userB}=0.999999231 [mEq/L] is obtained. By subtracting the volumes from the foregoing blood sodium concentrations I′BSC, the blood sodium concentrations SBSC [mEq/L] after discharge of sodium for the users A and B are calculated as follows:

SBSC_{userA}=I′BSC_{userA}−DSV_{userA}=159.008−0.999925453=158.0087702 [mEq/L]; and

SBSC_{userB}=I′BSC_{userB}−DSV_{userB}=168.157−0.999999231=167.157392 [mEq/L].

The physiological parameter calculation unit 13 outputs, as a simulation results, the calculated blood sodium concentrations SBSC that has been updated to the error calculation unit 14 and the equilibrium state calculation unit 15.

Subsequently, in step S105, the control unit 10 of the model optimization apparatus 1 determines whether it is a time for performing the calculation, that is, whether a condition “t mod cycleHour=0” is satisfied. As described above, in the example, the calculation is performed when the time t is a cycle of cycleHour. In particular, in the example, because cycleHour is 24, the calculation is performed when t is 24, 48, or 72. When “t mod cycleHour=0” is not satisfied in step S105 (NO), the control unit 10 of the model optimization apparatus 1 does not update the viscera parameter and the processing proceeds to step S109. When “t mod cycleHour=0” is satisfied (YES), the control unit 10 of the model optimization apparatus 1 determines that it is a time for performing the calculation and the processing proceeds to step S106.

In step S106, the error calculation unit 14 calculates an error between the sample data which is the actually measured value and the simulation result which is the estimated value. That is, the error calculation unit 14 calculates, as an error, a difference between the sample data and the calculation result received from the physiological parameter calculation unit 13.

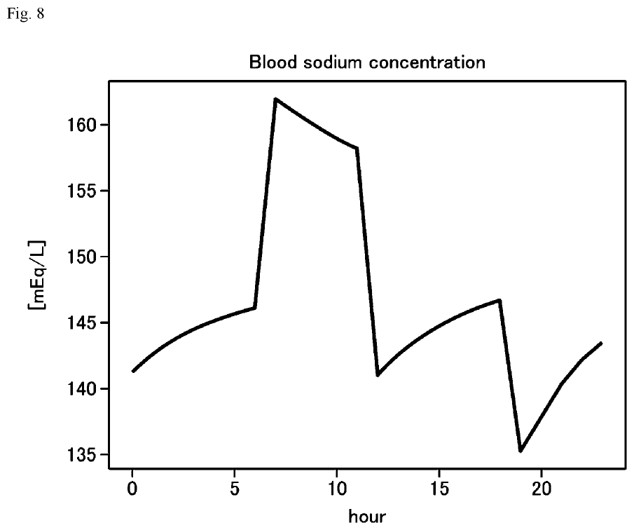

FIG. 8 illustrates an estimated value which is a simulation result of the blood sodium concentration in 0 to 24 hours of the user A. In the example, the error calculation unit 14 uses, as change characteristics of the physiological parameter, an average value ave_{BSC}, a maximum value max_{BSC}, and a minimum value min_{BSC} of the estimated values of the physiological parameter of each user in a cycleHour interval of a calculation target to calculate an error ER using the following equation.

ER=\sqrt{(ave_{BSC}−t_ave_{BSC})²}+\sqrt{(max_{BSC}−t_max_{BSC})²}+\sqrt{(min_{BSC}−t_min_{BSC})²} [Math. 4]

In the foregoing equation, t_ave_{BSC}, t_max_{BSC}, and t_min_{BSC} respectively indicate an average value, a maximum value, and a minimum value of the actually measured values measured from each user in the cycleHour interval. Here, in the example, since only one piece of measured data is used, the number of actually measured values is one. In the calculation of the foregoing error ER, the same actually measured value (the blood sodium concentration=140.4 of the sample) is used. The obtained average value ave_{BSC}, maximum value max_{BSC}, and minimum value min_{BSC} of the user A in the interval of 0 to 24, that is, a time (0, 24], are 146.603839, 161.962159, and 135.264642. At this time, the error ER is calculated using the following equation.

$$ER = \sqrt{{( 146.603839 - 140.4 )}^{2}} + \sqrt{{( 161.962159 - 140.4 )}^{2}} + \sqrt{{( 135.264642 - 140.4 )}^{2}} = 32.901356 \qquad [ Math . 5 ]$$

When values other than the blood sodium value are targets, the error calculation unit 14 adds errors of all the physiological parameters to calculate the error ER. The error ER calculated by the error calculation unit 14 is output to the equilibrium state calculation unit 15.

Subsequently, in step S107, the equilibrium state calculation unit 15 calculates the equilibrium state using the simulation result received from the physiological parameter calculation unit 13. When t is τ+cycleHour (where τ is an integer multiple of cycleHour), the equilibrium state at a time (τ, τ+cycleHour] is obtained by comparison with a simulation result at the immediately previous time (τ−cycleHour, τ]. Specifically, an equilibrium state ES is calculated as in the following equation.

ES=\sqrt{(c_ave_{BSC}−p_ave_{BSC})²}+\sqrt{(c_max_{BSC}−p_max_{BSC})²}+\sqrt{(c_min_{BSC}−p_min_{BSC})²} [Math. 6]

In the foregoing equation, c_ave_{BSC}, c_max_{BSC}, and c_min_{BSC} are respectively an average value, a maximum value, and a minimum value of the estimated values which are the simulation results, that is, the calculation results of the physiological parameter calculation unit 13, in the interval of the time (τ, τ+cycleHour]. Here, p_ave_{BSC}, p_max_{BSC}, and p_min_{BSC} are an average value, a maximum value, and a minimum value of the simulation result in the interval of the time (τ−cycleHour, τ]. Since the average value, the maximum value, and the minimum value are also calculated by the error calculation unit 14, the equilibrium state calculation unit 15 may receive and use the calculation results from the error calculation unit 14.

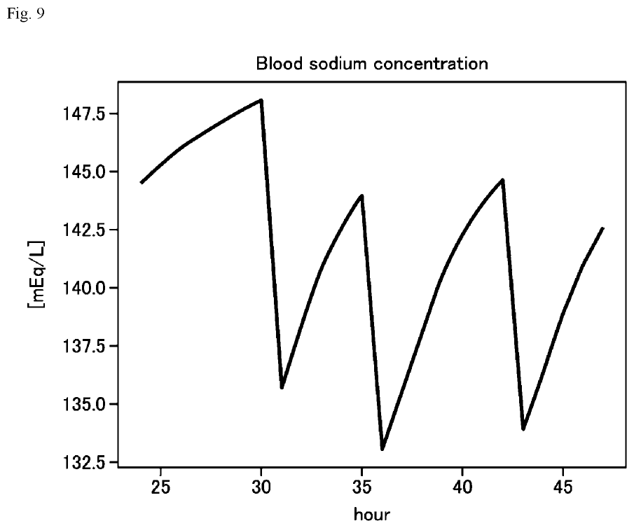

FIG. 9 illustrates an example of a simulation result of the blood sodium concentration in 24 to 48 hours of the user A.

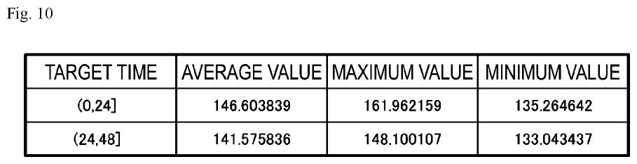

FIG. 10 illustrates an average value, a maximum value, and a minimum value of the blood sodium concentration in each target time interval obtained from the simulation results (the estimated values) of FIGS. 8 and 9.

For example, when the average value, the maximum value, and the minimum value are calculated at each target time as illustrated in FIG. 10, the equilibrium state ES in an interval of the time (24, 48] is obtained as in the following equation.

ES=\sqrt{(141.575836−146.603839)²}+\sqrt{(148.100107−161.962159)²}+\sqrt{(133.043437−135.264642)²}=36.726184 [Math. 7]

The equilibrium state calculation unit 15 obtains a sum value of the error ER and the equilibrium state ES as the degree of inappropriateness US.

The equilibrium state calculation unit 15 outputs the degree of inappropriateness US at the calculated time (τ, τ+cycleHour] to the viscera parameter updating unit 16.

In step S108, the viscera parameter updating unit 16 updates the viscera parameter based on the degree of inappropriateness US received from the equilibrium state calculation unit 15. The viscera parameter updating unit 16 determines the viscera parameter in a searching manner using the degree of inappropriateness US output from the equilibrium state calculation unit 15. The viscera parameter updating unit 16 can use, for example, a full searching scheme, but may use another updating method. The viscera parameters to be optimized are DSV_vol and DSV_vel, as described above.

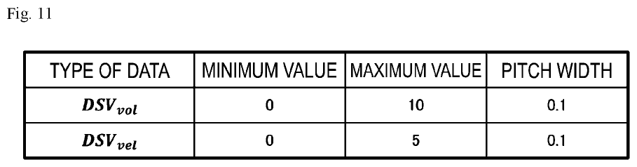

FIG. 11 illustrates an example of a searching area of the viscera parameters that can be used by the viscera parameter updating unit 16. As illustrated in FIG. 11, a viscera parameter set includes a minimum value, a maximum value, and a pitch value of each viscera parameter. The viscera parameter updating unit 16 obtains, for each viscera parameter set, the degree of inappropriateness US calculated by the equilibrium state calculation unit 15 and outputs a combination of the viscera parameters in which the degree of inappropriateness US is minimum as the viscera parameters of the target user.

In step S109, the control unit 10 of the model optimization apparatus 1 increases the time t(t=t+1) and the processing returns to step S102. As described above, when the time t exceeds maxHour in step S102, the processing ends.

### Effects

As described above in detail, in the model optimization apparatus 1 according to the embodiment of the present disclosure, the estimated value of the physiological parameter indicating the physiological state are calculated by the estimation model used to simulate physiological activity of an organ. Then, the error between the actually measured value and the estimated value of the physiological parameter and the difference in the estimated value between the given previous and subsequent time intervals are calculated, and the estimation model is adjusted in consideration of the error and the difference.

In this way, by considering the change characteristics of the estimated value in the given time interval, it is possible to incorporate the equilibrium state of the physiological parameter in an action of an individual user or an operation of the viscera function into the simulation. Accordingly, the model optimization apparatus 1 according to the embodiment can provide a simulation technology reflecting the physiological activity of an individual user more appropriately focusing on the equilibrium state in the physiological activity.

### Other Embodiments

The present disclosure is not limited to the foregoing embodiments. For example, the functional units included in the model optimization apparatus 1 may be distributed and disposed in a plurality of devices, and the devices may cooperate to perform the processing. Each functional unit may be implemented using a circuit. The circuit may be a dedicated circuit that implements a specific function or may be a general-purpose circuit such as a processor.

Further, the sequence of the above-described processes is not limited to the described sequence, and the order of several steps may be switched or several steps may be performed simultaneously in parallel. The series of above-described processing operations may not be necessarily continued chronologically and each step may be performed at any timing.

The above-described scheme can also be stored as a program (software) that can be executed by a computer, for example, in a recording medium (a storage medium) such as a magnetic disk (a floppy (trade name) disk, a hard disk, or the like), an optical disc (a CD-ROM, a DVD, an MO, or the like), a semiconductor memory (a ROM, a RAM, a flash memory, or the like) and can also be transmitted and distributed through a communication medium. The program stored in a medium also includes a setting program for loading software (including not only an execution program but also a table and a data structure) executed by a computer in the computer. A computer achieving the foregoing device reads the program recorded on a recording medium to construct software in accordance with the setting program in some cases, and the software controls an operation to perform the above-described processing. The recording medium mentioned in the present specification is not limited to a recording medium for distribution and includes a storage medium such as a magnetic disk and a semiconductor memory provided inside the computer or a device connected via a network.

In addition, the setting or the like of the parameters in the mathematical model can also be modified variously within the scope of the present disclosure without departing from the gist of the present disclosure.

In short, the present disclosure is not limited to the foregoing embodiments and can be modified variously in execution stages within the scope of the present disclosure without departing from the gist of the present disclosure. The embodiments may be appropriately combined and combined effects can be obtained in this case. Further, the foregoing embodiments include various disclosures and various disclosures can be extracted by a combination selected from a plurality of constituent elements. For example, even if several constituent elements are deleted from all the configuration elements in the embodiments, configurations from which these constituent elements are deleted can be extracted as the present disclosures when the problems can be solved and the effects can be obtained.

### REFERENCE SIGNS LIST

1 Model optimization apparatus

10 Control unit

11 Viscera model construction unit

12 User action calculation unit

13 Physiological parameter calculation unit

14 Error calculation unit

15 Equilibrium state calculation unit

16 Viscera parameter updating unit

20 Storage unit

21 Initial viscera parameter database, initial viscera parameter DB

22 User action database, user action DB

30 Input/output interface, Input/output I/F

40 Input device

50 Display device

1. An optimization apparatus comprising:
a processor; and
a storage medium having computer program instructions stored thereon, when executed by the processor, perform to:
estimate a physiological parameter indicating a physiological state using an estimation model;
to calculate a first difference value based on a first estimated value of the physiological parameter in a first time interval and an actually measured value of the physiological parameter in the first time interval;
calculate a second difference value based on the first estimated value and a second estimated value of the physiological parameter in a second time interval previous to the first time interval; and
adjust the estimation model based on the first difference value and the second difference value.

2. The optimization apparatus according to claim 1, wherein the computer program instructions further perform to adjusts the estimation model by re-calculating a model parameter of the estimation model such that a sum of the first difference value and the second difference value is minimized.

3. The optimization apparatus according to claim 2, wherein the computer program instructions further perform to
estimates, as the physiological parameter, a parameter related to intake or discharge of a substance in an organ, and
re-calculates, as the model parameter, a parameter related to a change in a concentration of the substance or a volume of the substance in the organ.

4. The optimization apparatus according to claim 3, wherein the computer program instructions further perform to acquire action information indicating a detail of an action of a subject and calculate an intake volume or a discharge volume of the substance of the subject based on the action information.

5. The optimization apparatus according to claim 1, wherein the computer program instructions further perform to calculates the first difference value as a total sum of absolute differences between an average value, a maximum value, and a minimum value of the physiological parameter estimated for the first time interval and an average value, a maximum value, and a minimum value of the physiological parameter actually measured for the first time interval.

6. The optimization apparatus according to claim 1, wherein the computer program instructions further perform to calculates the second difference value as a total sum of absolute differences between an average value, a maximum value, and a minimum value of the physiological parameter estimated for the first time interval and an average value, a maximum value, and a minimum value of the physiological parameter estimated for the second time interval.

7. An optimization method performed by a computer, the method comprising:
estimating a physiological parameter indicating a physiological state using an estimation model;
calculating a first difference value based on a first estimated value of the physiological parameter in a first time interval and an actually measured value of the physiological parameter in the first time interval;
calculating a second difference value based on the first estimated value and a second estimated value of the physiological parameter in a second time interval previous to the first time interval; and
adjusting the estimation model based on the first difference value and the second difference value.

8. A non-transitory computer-readable medium having computer-executable instructions that, upon execution of the instructions by a processor of a computer, cause the computer to function as the optimization apparatus according to claim 1.
